(19) United States
(12) Patent Application Publication
(10) Pub. No.: US 2024/0216433 A1
(43) Pub. Date: Jul. 4, 2024

(54) METHODS AND SYSTEMS FOR PREPARATION OF MONONUCLEAR-PLATELET RICH FIBRIN MATRIX, AND COMPOUNDS THEREOF

(71) Applicant: PRP Concepts, Inc., Middletown, NJ (US)

(72) Inventor: Richard J. CARROLL, Syracuse, NY (US)

(21) Appl. No.: 18/562,956

(22) PCT Filed: May 12, 2022
(86) PCT No.: PCT/US2022/028941

§ 371 Date: Nov. 21, 2023

Related U.S. Application Data
(60) Provisional application No. 63/193,889, filed on May 27, 2021.

(51) Int. Cl. A61K 35/19; A61J 1/05; A61K 35/15; A61K 35/17; A61M 1/02

(52) U.S. Cl. CPC A61K 35/19; A61J 1/05; A61K 35/15; A61K 35/17; A61M 1/029

(57) ABSTRACT

Provided is a purified non-naturally occurring wound healing composition, comprising platelets, monocytes, and lymphocytes, wherein said composition is substantially free of neutrophils. Also provided are a system of preparing said purified non-naturally occurring wound healing composition: a method of making the composition; and a method of using the composition to treat wounds.

### FIELD OF THE INVENTION

The present invention generally relates to blood cell processing techniques and apparatus. In one embodiment, the present invention relates to methods and systems for separating platelets and mononuclear cells, such as lymphocytes and monocytes, together from whole blood specimens, and specifically relates to blood separation methods and systems which provide a high recovery of platelets and mononuclear cells without significant contamination by red blood cell or by polymorphonuclear granulocytes.

All publications, patents, patent applications, and other references cited in this application are incorporated herein by reference in their entirety for all purposes and to the same extent as if each individual publication, patent, patent application or other reference was specifically and individually indicated to be incorporated by reference in its entirety for all purposes. Citation of a reference herein shall not be construed as an admission that such is prior art to the present invention.

### BACKGROUND OF THE INVENTION

Acute wounds follow an organized wound healing sequence and often heal between 3 and 4 weeks. When a wound is still present 4 weeks after wounding, it is defined as a chronic wound. [Demidova-Rice, T N, et al. (2012) Adv Skin Wound Care. 25(7): 304-14]. Many research studies have been conducted on chronic wound management to address the rising demand for effective and affordable care. The healing trajectory of chronic wounds is expected to take 12 weeks [Sibbald, R G, et al. (2011) Adv Skin Wound Care. 24: 415-37]. This period may be prolonged if the wound presents with an altered molecular environment, chronic inflammation, or fibrosis [Cañedo-Dorantes, L, et. al. (2019) Int J Inflam. 2019: 3706315], or uncorrected preexisting systemic factors.

Chronic wounds, the most common of which are diabetic foot ulcers (DFU), pressure ulcers (PU), venous leg ulcers (VLU), and nonhealing surgical wounds, are a major healthcare problem. Chronic wounds usually occur in older individuals with underlying conditions such as diabetes mellitus, vascular disease, and obesity [Gould, L, et al. (2015) Wound Repair Regen. 23(1): 1-13]. Compromised immune and nutritional status as well as chronic mechanical stress have also been shown to contribute to poor wound healing outcomes [Eming, S A, et al. (2014) Sci Transl Med. 6(265): 265sr6]. Chronic wounds are associated with alarmingly high mortality: the 5-year mortality rates of ischemic (55% mortality rate), neuropathic (45%), and neuroischemic (18%) diabetic foot ulcers [Moulik, P K, et al. (2003) Diabetes Care. 26(2): 491-4], are higher than or similar to those associated with breast cancer and prostate cancer (18% and 8%, respectively) [Armstrong, D G, et al. (2007) Int. Wound J. 4(4): 286-287]. Chronic wounds are also associated with high healthcare costs: in the USA, total spending estimates for chronic nonhealing wounds ranged from US$28.1B to US$96.8B in 2014 according to a retrospective analysis of the Medicare 5% Limited Data Set [Nussbaum, S R, et al. (2018) Value Health. 21(1): 27-32]. Despite the alarming prevalence and high costs of care, efficient treatments are still lacking.

The complexity and multiplicity of the diabetic foot wound for example, makes it an immensely challenging therapeutic target, and the lopsided progress seen in murine models highlights the need for new methods to overcome the bench-to-bedside barrier. Clinical progress requires more innovative research strategies that harness both the existing knowledge and the potential of new advances across disciplines [Barakat, M, et al. (2020) Adv Wound Care. https://doi.org/10.1089/wound2020.1254].

Blood platelets are small bioactive anuclear cells with diameters that vary between 2 and 4 μm and are derived from mature megakaryocytes in the bone marrow and lungs [Lefrançais, E, et al. (2017) Nature. 544(7648): 105-109]. Platelets are essential for primary hemostasis, but they also play important roles in tissue regeneration and inflammation [Etulain J. (2018) Platelets. 29(6): 556-568].

Platelet α-granules constitute the major granule population in terms of size and number within the platelet. They contain adhesion and growth factors, such as transforming growth factor-β (TGF-β), platelet-derived growth factor (PDGF), platelet-derived endothelial cell growth factor (ECGF), vascular endothelial growth factor (VEGF), basic fibroblast growth factor (bFGF), epidermal growth factor (EGF), and insulin-like growth factor (IGF) as well as P-selectin, platelet factor 4, fibronectin, Beta-thromboglobulin, von Willebrand Factor (vWF), fibrinogen, and coagulation factors V and XIII [Eisinger, F, et al. (2018) Front Med. 5: 317].

Platelets have been used in wound care for several decades. The benefits of platelet rich plasma (PRP) administration are associated with an economical advantage, taking into consideration that PRP administration does not require complex equipment or training for its execution. Moreover, due to their primary autologous origin, concerns of disease transmission or immunogenic reactions can be disregarded [Etulain, J. (2018) Platelets. 29(6): 556-568]. Platelets are easily available in large quantities from blood. A normal platelet count ranges from 150,000 to 450,000 platelets per microliter of blood. A platelet has 50-80 alpha granules which release hundreds of bioactive proteins [Blair, P, et al. Blood Rev. (2009) 23(4): 177-189] including growth factors, adhesion molecules, and serotonin, which promotes cellular viability, proliferation, and migration [Cloutier, N, et al. (2018) PNAS 115(7): E1550-E1559]. Platelet-derived microparticles (PDMs) stimulate the release of cytokines, activate intracellular signaling pathways, promote angiogenesis, and are involved in tissue regeneration [Neumüller, J, et al. In: Khan M, ed. Intechopen. London: Intechopen; (2015). pp. 255-284]. Platelets interact with immune cells [Hu H, et al. Thromb Haemost (2010) 104: 1184-1192] and have analgesic effects [Miyamoto, H, et al. J Oral Max Surgery, Med, and Path. (2020) 32(4): 237-240]. Platelets are involved in tissue remodeling [Langer H F, et al. Arterioscler Thromb Vasc Biol. (2007) 27: 1463-1470] and recruit bone marrow derived progenitor cells [Massberg S, et al. J Exp Med. (2006) 203: 1221-1233] and mesenchymal stem cells [Langer H F, et al. J Mol Cell Cardiol. (2009) 47: 315-325]. Platelets are also important for the maintenance of vascular integrity [Boulaftali Y, et al. J Clin Invest. (2013) 123: 908-916]. Platelet mediators stimulate extracellular matrix formation and connective tissue restructuring [Xu X, et al. Cells Tissues Organs. (2013) 197: 103-113].

For the treatment of chronic non-healing cutaneous wounds, technology has been developed to insert concentrated platelets into the wound to speed the wound healing process. Platelets isolated from the peripheral blood are an autologous source of growth factors. The term platelet-rich plasma (PRP) was introduced in the 1970s to describe the autologous preparations and enrichment of platelets in a plasma concentrate [Pietrzak, W S, et al. (2005) J Craniofac Surg. 16: 1043-1054]. PRP, also known as autologous conditioned plasma, is a concentrate of platelet-rich plasma derived from whole blood, centrifuged to remove red and white blood cells. Autologous PRP gel consists of cytokines, growth factors, chemokines, and a fibrin scaffold derived from a patient's blood [Frykberg, R G, et al. (2010) Ostomy Wound Manage. 56(6): 36-44]. The mechanism of action for PRP gel is thought to be the molecular and cellular induction of normal wound healing responses similar to that seen with platelet activation.

Monocytes typically circulate through the blood for 1 to 3 days before migrating into tissues, where they become macrophages or dendritic cells. Macrophages exhibit plasticity and adopt pro-inflammatory, pro-wound-healing, pro-fibrotic, anti-inflammatory, anti-fibrotic, or tissue-regenerating phenotypes. According to the activation state and functions of macrophages, they can be divided into M1-type (classically activated macrophage) and M2-type (alternatively activated macrophage). The balance between M1 and M2 macrophages plays an important role in wound healing [Mosser D M, et al. (2008) Nature Rev Immunol. 8(12): 958-969]. Most well characterized, the M1 initiates the pro-inflammatory immune responses to pathogen colonization [Porta, C, et al. (2015) Semin Immunol. 27(4): 237-248]. As the inflammation abates, these alarmins are believed to facilitate the onset of anti-inflammatory or wound healing responses characterized by the presence of alternatively activated macrophages, or the M2 phenotypes.

Interleukin 4 (IL-4) produced by T helper type 2 lymphocytes (Th2) cells can convert macrophages into M2-type macrophages that inhibit inflammation [Abramson, S L, et al. (1990) J Immunol. 144(2): 625-630]. M2 macrophages mainly secrete anti-inflammatory cytokines, which have the function of reducing inflammation and play an important role in wound healing and tissue repair.

Metabolic pathway utilization shifts characterize macrophage polarization with resulting metabolic and immune outcomes impacting host-pathogen interactions during wound healing [Anders, C B, et al. (2019). Curr Opinion Infect Dis. 32(3): 204-209]. Macrophage plasticity is critical for normal tissue repair to ensure transition from the inflammatory to the proliferative phase of healing.

Studies in mice have shown that a specific combination of growth factors (GFs) enhances the survival, adhesion, and angiogenic potential of mononuclear cells [Jin, E, et al. (2013) J Cell. Mol. Med. 17(12): 1644-1651]. In vivo wound healing results revealed that GF-treated wounds demonstrated accelerated wound healing at days 7 and 14 compared with those untreated. The histological analyses demonstrated that the number of engrafted cells and transdifferentiated keratinocytes in the wounds were significantly higher in the GF-treated subjects. This suggests that priming of mononuclear cells with growth factors released by platelets can enhance cell-based therapies.

Macrophages enter a wound and produce IL-10, which can then cause the cells that are around the wound to start closing the wound [Quiros, M, et al. (2017) J Clin Invest. 127(9): 3510-3520]. Studies of mucosal wounds defined some of the signaling pathways that IL-10 uses to orchestrate wound repair.

Lymphocytes have a role in regulating the direction of wound repair, with or without scaring. T lymphocyte subsets have been shown in mice to attenuate the degree of inflammation and promote relevant neovascularization, thereby reducing the risk of dermal scarring [Wang, λ, et al. (2019) Adv in Wound Care 8(11): 527-537]. The time course of T lymphocyte infiltration into the wound shows that CD3+ T lymphocytes are present in the wound at day 3, peak at day 14, and persist until day 30, suggesting a significant T lymphocyte role in dermal wound healing and scarring responses. CD4+ T lymphocytes may represent the key lymphocyte population that regulates the responses to wound injury and repair. There is a balance between inflammation and angiogenesis directed by T lymphocytes that may be a part of the mechanisms that account for tissue repair and scar formation.

Addition of mononuclear cells (lymphocytes and monocytes) to platelets can enhance wound healing. The immune system plays an integral role in successful wound healing. In addition to contributing to host defenses, immune cells are critical regulators of wound healing through the secretion of cytokines, lymphokines, and growth factors [Park, J E, et al. Am J Surg. (2004) 187(5): S11-S16]. Various studies of PRP technologies reveal that the wide variation of blood components, including platelets, red blood cells, leukocytes, pH, and glucose in PRP extractions play an important role in successful wound healing. The high concentrations of cells are important, as are the white blood cell count in PRP samples. The non-standardized method of recovering a PRP fraction has frequently been ignored by investigators, being considered insignificant. However, the lack of standardization of PRP preparations for clinical use has contributed at least in part to the varying clinical efficacy in PRP use [Fitzpatrick, J, et al. (2017) Orthop J Sports Med. 5(1):2325967116675272].

Various anticoagulants have been used in blood collection/separation devices either alone or in conjunction with a cell-sustaining solution in order to preserve the blood sample in an uncoagulated state for a period of time prior to centrifugation and analysis. For example, some common anticoagulants include sodium heparin, K₂EDTA, K₃EDTA, and various concentrations of sodium citrate. In particular, sodium citrate solutions have been used for many years as anticoagulants. U.S. Pat. No. 5,494,590, incorporated by reference, discloses a sodium citrate-based anticoagulant solution having a pH ranging from above pH6.0 to about pH8.5 and a sodium citrate concentration preferably ranging from about 0.05M to about 0.2M.

It is known that calcium plays a key role in the blood coagulation cascade. Sodium citrate solutions prevent the participation of calcium in blood coagulation. Typically, these sodium citrate solutions are added to freshly collected whole blood to prevent coagulation. Subsequently, calcium can be added back to the whole blood suspension to induce subsequent coagulation when desired. Sodium citrate is a particularly advantageous anticoagulant as it provides good buffering capabilities over a range of pH. In particular, the buffering capability of sodium citrate is attributable to three carboxyl groups present on the corresponding acid of the compound. Since sodium citrate is the corresponding sodium-based salt of citric acid, it is the citric acid/sodium citrate combination that functions to perform the buffering chemistry.

As mentioned above, citric acid (hydroxytricarboxylic acid) has 3 carboxyl groups and consequently 3 pKa's. The first pKa₁ appears at a pH of about 3.06. The second pKa₂ appears at a pH of about 4.76. The third pKa₃ appears at a pH of about 5.4. Accordingly, sodium citrate performs its most effective buffering functions at these pH values and is especially useful in performing buffering functions when added to in vitro cell suspensions. Consequently, sodium citrate has been used as an anticoagulant in a variety of blood separation devices due to its buffering capability over a range in pH. Citrate has been commonly used as an anticoagulant in three types of solutions. The first type of solution is referred to as buffered sodium citrate. The second type of solution is typically referred to as CPD solution or citrate-phosphate-dextrose. The third type is denoted as ACD or acid-citrate-dextrose. The citrate ion concentration in these solutions is typically greater than the concentration needed to perform an anticoagulation function.

U.S. Pat. No. 4,640,785, incorporated by reference, discloses a method for the separation of lymphocytes and monocytes from blood samples. An integral part of the invention is an improved blood separation tube utilizing a gel-like substance having a specific gravity between 1.060-1.065 g/cm³ to significantly enhance the purity of cell separation, while providing acceptable cell yields.

U.S. Pat. No. 4,816,168, incorporated by reference, discloses a method for inhibiting the apparent shift in the buoyant density of and/or restore any loss in the buoyant density of the granulocytic white blood cells in a sample of blood, thereby ensuring the quality of the separation of lymphocytes and monocytes from granulocytes in a blood sample.

U.S. Pat. No. 6,368,298, incorporated by reference, discloses a method of preparing a solid-fibrin web. The method includes drawing blood from a patient, separating plasma from the blood, contacting the plasma with a calcium-coagulation activator and concurrently coagulating and centrifuging the plasma to form the solid-fibrin web. The solid-fibrin web is suitable for regenerating body tissue in a living organism.

U.S. Pat. No. 6,979,307, incorporated by reference, discloses a system for preparing an autologous solid-fibrin web suitable for regenerating tissue in a living organism. The system includes a sealed primary container containing a separation medium and a low-density high-viscosity liquid.

U.S. Pat. No. 7,745,106, incorporated by reference, discloses methods and devices for preparing a solid-fibrin web. One method may include drawing blood from a patient, separating plasma from the blood, contacting the plasma with a calcium-coagulation activator and concurrently coagulating and axially centrifuging the plasma to form the solid-fibrin web. The solid-fibrin web may be suitable for regenerating body tissue in a living organism.

U.S. Pat. No. 8,802,362, incorporated by reference, discloses methods and devices for preparing a solid-fibrin web. One method may include drawing blood from a patient, separating plasma from the blood, contacting the plasma with a calcium-coagulation activator and concurrently coagulating and axially centrifuging the plasma to form the solid-fibrin web. The solid-fibrin web may be suitable for regenerating body tissue in a living organism.

U.S. Pat. No. 8,491,564, incorporated by reference, discloses a system for preparing an autologous solid-fibrin web suitable for regenerating tissue in a living organism. The system includes a sealed primary container containing a separation medium and a low-density high-viscosity liquid.

U.S. Pat. No. 10,617,812, incorporated by reference, discloses a system for obtaining plasma enriched in platelets which is closed to the atmosphere. The system includes a collection tube containing an anticoagulant portion and a separation gel.

None of the systems prepare a concentrated mononuclear (monocytes and lymphocytes) cell preparation with platelets in plasma using a thixotropic gel separator device and a density gradient media without the use of thrombin or batroxobin. The advantage of combining mononuclear cells with platelets for wound healing combines the immune function of the mononuclear cells with the cell signaling of the platelets. Granulocytes are excluded since they contribute to inflammation. The use of PRP rich in neutrophils could result in a higher collagen type III to collagen type I ratio, adding to fibrosis and decreased tendon strength [Zhou, Y, et al. (2016) BioMed Res. Int. 2016: 1-8]. Other neutrophil-mediated deleterious properties include the release of inflammatory cytokines and matrix metalloproteinases (MMPs) that promote pro-inflammatory and catabolic effects when applied to tissues [Fedorova, N V, et al. (2018) Mediat Inflamm. 2018: ID 1574928]. Neutrophils can produce extracellular traps (NETs), large extracellular web-like structures composed of decondensed chromatin bound to various cytosolic and granule proteins. While originally recognized as a defense mechanism against pathogens, they can hinder regeneration [Wong, S L, et al. (2015) Nat Med. 21(7):815-819]. In addition to the presence of neutrophils shortly after wounding, neutrophils remain within the wound after the NET barrier is reestablished. Taken together, study results demonstrate that although neutrophils are stimulated by a common pro-regenerative cue, their presence and NETs can hinder regeneration [Wier, E, et al. bioRxiv (2020.07.06). 189910].

A need exists in the art, therefore, for a purified non-naturally occurring wound healing composition, comprising platelets, monocytes and lymphocytes, wherein said composition is substantially free of neutrophils. Such a composition would be useful, for example, for wound healing in a subject in need thereof.

### SUMMARY OF THE INVENTION

In an embodiment of the invention, provided is a purified non-naturally occurring wound healing composition, comprising platelets, monocytes and lymphocytes, said composition being substantially free of neutrophils. In a further embodiment, the purified non-naturally occurring wound healing composition is a mononuclear-platelet rich fibrin matrix.

Also provided are a system for preparing a purified non-naturally occurring wound healing composition, comprising platelets, monocytes and lymphocytes, said composition being substantially free of neutrophils, as well as a method of preparing such a composition using the system of the invention.

Further provided is a method of treating a wound, comprising the step of administering the purified non-naturally occurring wound healing composition of the invention which comprises platelets, monocytes and lymphocytes, said composition being substantially free of neutrophils, to a subject in need thereof.

### BRIEF DESCRIPTION OF THE FIGURES

FIG. 1. illustrates an exemplary sodium citrate-based anticoagulant solution deployed within a blood collection tube and separation assembly.

FIG. 2. illustrates an exemplary transfer device between a processed blood collection tube and the centrifugation assembly for clot formation.

### DETAILED DESCRIPTION OF THE INVENTION

It is to be understood that the figures and descriptions of the present invention have been simplified to illustrate elements that are relevant for a clear understanding of the present invention, while eliminating, for the purpose of clarity, many other elements found in typical pharmaceutical compositions and methods of stabilization. Those of ordinary skill in the art will recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art. Furthermore, the embodiments identified and illustrated herein are for exemplary purposes only and are not meant to be exclusive or limited in their description of the present invention.

The current invention generally provides for the separation of mononuclear cells (monocytes and lymphocytes) and platelets from whole blood by centrifugation. In one embodiment, the invention is a blood collection tube that utilizes a non-Newtonian, thixotropic gel of a specific density. This gel is positioned within the blood collection tube in order to form a stable barrier between a liquid density medium, such as Ficoll® Paque [Sigma Aldrich], placed below the gel barrier, and a liquid anticoagulant, preferably sodium citrate, placed above the gel barrier. The evacuated blood collection tube allows the collection of a blood sample using standard venipuncture prior to centrifugation. The blood mixes with the anticoagulant upon blood draw to prevent coagulation of the blood. Upon centrifugation, the blood components are separated by their cell density, allowing the denser red blood cells and granulocyte populations to migrate below the gel barrier, while the less dense mononuclear cells and platelets remain above the gel barrier. Effective separation and isolation of these cells is often critical to various clinical assays as well as to research laboratory protocols. Therapeutic application of the isolated cell fractions is also of importance, such as the use of platelet rich plasma in wound care.

Once the separation has occurred, one application involves the direct injection of the mononuclear-platelet plasma suspension into the injured site. The upper fraction of mononuclear cells, platelets, and plasma, i.e., the mononuclear-platelet rich plasma (“M-PRP”) is aseptically transferred to an injection device, such as a needle and syringe. The platelets collected in PRP are activated by the addition of calcium gluconate or calcium chloride, as an example, which induces the release of factors from alpha granules. The process increases the concentration of mononuclear cells and platelets and the concentrated M-PRP is then injected into and around the affected area, jump-starting and significantly strengthening the body's natural healing signals.

A similar application is the use of the M-PRP suspension as a glue for split thickness skin grafts. This helps fasten the graft to the substrate and the cells contained within the M-PRP speed up the “take” of the graft. This use also eliminates the need for stiches or staples to fasten the graft to the substrate.

In another application, once the separation has occurred, the upper fraction of mononuclear cells, platelets, and plasma is aseptically transferred to a second evacuated tube or vial containing calcium chloride. Upon contact, the calcium ions overcome the anticoagulant effect of the citrate and causes the plasma fibrinogen to activate to fibrin, causing the cell suspension to be trapped in a fibrin clot. Behind this clot formation, there is the intrinsic coagulation pathway, which is activated at the level of factor XII by the tube glass surface and proceeds in the presence of calcium to convert prothrombin to thrombin, subsequently fibrinogen to fibrin, and consequently facilitates fibrin polymerization and cross-linking [Margolis, J. (1956) Nature. 178: 805-806].

The mechanism of Ca²⁺-induced clot formation includes a fibrin mesh deposited on platelet aggregates in a white thrombus. Platelet aggregates function like nuclei of clot formation and are located mainly near the center or in a deep region of a clot, and this clot may be classified essentially as a white thrombus. In this case, growth factors stored in platelet a granules can be assumed to be retained for a relatively long time. This type of clot functions as a long-lasting carrier of the growth factors with a better regenerative potential.

As clot formation proceeds, the second evacuated tube or vial is centrifuged to advantageously produce a mononuclear-platelet rich fibrin matrix (“M-PRFM”), which can be placed directly on a wound following centrifugation. This fibrin matrix allows easier handling on the M-PRFM and can be sutured into place if required, as in certain orthopedic applications.

In another application, once the separation has occurred, the upper fraction of mononuclear cells, platelets, and plasma is aseptically transferred to a second evacuated vial containing calcium chloride. This vial can have a flat bottom, which upon centrifugation, forms the M-PRFM into a flat membrane. This M-PRFM membrane has a greater surface area and thus there is greater exposure of the wound bed to the M-PRFM.

### DESCRIPTION OF CERTAIN EMBODIMENTS

A “patient” is a mammal, e.g., a human, mouse, rat, guinea pig, dog, cat, horse, cow, pig, or non-human primate, such as a monkey, chimpanzee, baboon or rhesus monkey, and the terms “patient” and “subject” are used interchangeably herein.

The term “treating”, with regard to a subject, refers to improving at least one symptom of the subject's disorder. Treating can be curing, improving, or at least partially ameliorating the disorder. The term “administer”, “administering”, or “administration” as used in this disclosure refers to administering a composition of the invention to the subject.

In one embodiment, provided is a fibrin matrix having an autologous fibrin molecular structure, which embeds mononuclear cells (monocytes and lymphocytes) and platelets, and acts as a biodegradable scaffold for supporting cell migration and the accomplishment of microvascularization. This advantageous mononuclear-platelet rich fibrin matrix (M-PRFM) acts as a delivery system of cells and growth factors leading to the enhancement of wound healing. The M-PRFM contains most of the platelets and mononuclear cells present in the initial blood specimen. Platelets are mostly activated and act to reinforce the strongly polymerized fibrin matrix. Mononuclear cells (monocytes and lymphocytes) are also trapped within the fibrin network and contribute to the tissue-healing process. The large quantities of mediators, particularly platelet growth factors, are released into the wound to activate the tissue regeneration process.

The M-PRFM may operate as a delivery system of cells and growth factors leading to enhancement of wound healing during the two first weeks. Platelets are mostly activated and act as a cement to reinforce the strongly polymerized fibrin matrix. Mononuclear cells are also trapped into a strong natural fibrin matrix. The cell composition of the M-PRFM implies that this biological material is a blood-derived living tissue and must be handled carefully to keep its cellular content alive and stable. During the formation of the M-PRFM, fibrils undergo lateral associations and form branches that result in a complex fiber network. The high degree of equilateral fibril branching results in the membrane elasticity. The fine nanostructure of fibrin, after the gel point, has been physicochemically characterized to show dynamic behavior and complex hierarchy at different scales.

The intricate architecture of the M-PRFM may offer advantageous mechanical behavior due to design and elasticity provided by the cross-linked monomer units. The morphology and mechanical behavior of the M-PRFM depend on the proportion of fibrinogen and thrombin. For instance, a low concentration of thrombin results in clots with thick fibers, less branch structures, and larger voids, thus less stable. Fibrin fiber diameter affects the surface area available for cell adhesion and interactions during platelet activation. An analysis of an exemplary fibrin network formed in accordance with an embodiment of the invention when using standard protocols produced M-PRFMs having a dense network of fibers of about 90 nm thickness. The microspaces found in the fibrin network are filled by cells and growth factors.

Furthermore, detected cross-linking between fibrin fibers mechanically stabilizes the architecture of fibrin networks and controls the fibrinolytic activity of plasmin. Fibrin not only acts as a scaffold into which cells infiltrate but also provides molecular signals to direct cell function, since it contains binding sites for integrins, growth factors, and other extracellular matrix components including fibronectin. The M-PRFM uses all the fibrinogen available in the plasma to convert to fibrin, thus ensuring the maximum fibrin density. Overall, the quality and quantity of fibrin fibers, in addition to growth factors, affect the potency and efficacy of M-PRFM in tissue healing

In another embodiment, provided is a system for preparing a purified wound-healing composition comprising platelets, monocytes and lymphocytes, the system comprising:
a first container having a sealable open end for receipt of a blood sample, and a closed end opposite the open end;
the first container containing a density separation medium disposed at a first position proximate the closed end, and a thixotropic gel having a density of approximately 1.055 to 1.080 g/cm³ disposed at a second position between the open end and the first position; and an anticoagulant solution having a pH in the range of 6.0 and 8.5 at a third position between the open end and the second position,
wherein the first container is useable to produce an extractable suspension of separated platelets, monocytes and lymphocytes and platelets from the blood sample when centrifuged; and
a second container having a sealable open end for receipt of an extracted suspension of separated platelets, monocytes and lymphocyte and plasma produced in the first container, the second containing a coagulation-activator,
wherein the second container is useable to produce the purified wound-healing composition from the suspension of separated platelets, monocytes and lymphocytes when centrifuged.

In one embodiment, the system further comprises a transfer device adapted for coupling with the first container open end, and coupling with second container open end, wherein the transfer device when coupled to the first container open end creates a sterile seal for receipt of the suspension of separated platelets, monocytes and lymphocytes, and plasma from the first container, and when coupled to the second container open end creates a sterile environment for transfer of the suspension of separated platelets, monocytes and lymphocytes, and plasma to such second container.

The density separation medium of the system comprises at least one of a non-Newtonian gel and a Newtonian liquid. In one embodiment, the density separation medium comprises an ionic substance having a molecular weight of less than about 1500. In another embodiment, the density separation medium is selected from the group consisting of sodium diatrizoate, derivatives thereof and combinations thereof. In a further embodiment, the density separation medium is selected from the group consisting of a polymer of sucrose or epichlorohydrin having a molecular weight of at least 400,000 and derivatives and combinations thereof.

In one embodiment, the system further comprises a first closure device for sealing the open end of the first container. The first closure device is adapted for vacuum sealing said open end of the first container. In one embodiment first closure device is pierceable by a cannula for supplying the blood sample to the first container by pressure differential.

In one embodiment, the system further comprises a second closure device for sealing the open end of the second container. In another embodiment, the second closure device is adapted for vacuum sealing said open end of the second container. In one embodiment, the second closure device is pierceable by a cannula for supplying the suspension of separated platelets, monocytes and lymphocytes, and plasma to the second container by pressure differential.

In another embodiment of the system, the purified wound-healing composition comprising platelets, monocytes and lymphocytes is a mononuclear-platelet rich fibrin matrix.

In one embodiment, the system further comprises a centrifuge adapted to receive at least one of the first and second containers.

In a further embodiment, the thixotropic gel of the system has a specific gravity of between 1.060 to about 1.065 g/cm³. In another embodiment, the separation medium has a specific gravity between 1.065 to about 1.085 g/cm³, and preferably a specific gravity of about 1.070 to about 1.080 g/cm³, and an optimal specific gravity of 1.077 g/cm³.

In another embodiment of the system, the pH is from about 6.5 to about 7.5 and preferably 6.85 to about 7.15, and an optimal pH of 7.0.

In one embodiment, the concentration of sodium citrate anticoagulant is from about 0.05M to about 0.20M, with a preferred concentration of sodium citrate from about 0.08M to about 0.13M, with an optimal sodium citrate concentration range from about 0.09M to about 0.11M. In one embodiment, the pH is 7.0 and the concentration of sodium citrate to 0.1M.

In a further embodiment, the anticoagulant comprises per liter 0.10 Molar 294 gm Sodium Citrate·2H₂O, 0.27 gm Citric Acid·H₂O; and pH 7.0. In one embodiment, the coagulation-activator is a calcium chloride (CaCl₂·2H₂0) clot activation solution at a concentration of between 0.05M to 0.3M, preferably between 0.1M to approximately 0.25M, with an optimal concentration of 0.2M.

In one embodiment, also is provided a system for preparing a purified wound-healing composition comprising platelets, monocytes and lymphocytes, the system comprising:
a first container having a sealable open end for receipt of a blood sample, and a closed end opposite the open end;
the first container containing a first density separation medium disposed at a first position proximate the closed end, and a second density separation medium disposed at a second position between the open end and the first position; and an anticoagulant solution having a pH in the range of 6.0 and 8.5 at a third position between the open end and the second position,
wherein the first container is useable to produce an extractable suspension of separated platelets, monocytes and lymphocytes, and plasma from the blood sample when centrifuged; and
a second container having a sealable open end for receipt of an extracted suspension of separated platelets, monocytes and lymphocytes, and plasma produced in the first container, the second containing a coagulation-activator,
wherein the second container is useable to produce the purified wound-healing composition from the suspension of separated platelets, monocytes and lymphocytes, and plasma when centrifuged.

In one embodiment of this system, the second density separation medium is a thixotropic gel having a density of approximately 1.055 to 1.080 g/cm³. In another embodiment, the first density separation medium comprises at least one of a Newtonian liquid. In a further embodiment, the first density separation medium comprises an ionic substance having a molecular weight of less than about 1500. The first density separation medium can be selected from the group consisting of sodium diatrizoate, derivatives thereof and combinations thereof.

In another embodiment of the system, the first density separation medium is selected from the group consisting of a polymer of sucrose or epichlorohydrin having a molecular weight of at least 400,000 and derivatives and combinations thereof.

In one embodiment of the invention, provided is a system wherein the purified wound-healing composition comprising platelets, monocytes and lymphocytes is a mononuclear-platelet rich fibrin matrix.

In another embodiment of the invention, provided is a purified non-naturally occurring wound healing composition, comprising platelets, monocytes and lymphocytes, wherein said composition is substantially free of neutrophils. In one embodiment, the concentration of neutrophils is less than 5% of the separated white blood cells.

Also provided as an embodiment of the invention is a method of producing a purified non-naturally occurring wound healing composition comprising platelets, monocytes and lymphocytes, and plasma from a sample of blood, comprising the steps of:
introducing said sample of blood into a first container having a sealable open end for receipt of said sample, and a closed end opposite the open end, said first container containing a density separation medium disposed at a first position proximate the closed end, and a thixotropic gel having a density of approximately 1.055 to 1.080 g/cm³ disposed at a second position between the open end and the first position; and an anticoagulant solution having a pH in the range of 6.0 and 8.5 at a third position between the open end and the second position;
centrifuging said first container to produce an extractable suspension of separated platelets, monocytes and lymphocytes, and plasma from the blood sample;
extracting said extractable suspension of separated platelets, monocytes and lymphocytes, and platelets into a second container containing a coagulation-activator and having a sealable open end for receipt of said extracted suspension of separated platelets, monocytes and lymphocytes, and plasma produced in the first container; and
centrifuging said second container to produce the purified non-naturally occurring wound healing composition comprising platelets, monocytes and lymphocytes.

In one embodiment of this method, the wound healing composition is substantially free of neutrophils. In another embodiment, the concentration of neutrophils is less than 5% of the separated white blood cells.

In a further embodiment of this method, the separation medium comprises at least one of a non-Newtonian gel and a Newtonian liquid. In one embodiment, the density separation medium comprises an ionic substance having a molecular weight of less than about 1500. In one embodiment, the density separation medium is selected from the group consisting of sodium diatrizoate, derivatives thereof and combinations thereof. For example, the density separation medium is selected from the group consisting of a polymer of sucrose or epichlorohydrin having a molecular weight of at least 400,000 and derivatives and combinations thereof.

In one embodiment, the method provides for a purified wound-healing composition comprising platelets, monocytes and lymphocytes that is a mononuclear-platelet rich fibrin matrix. In one embodiment, the thixotropic gel of the method has a specific gravity of between 1.060 to about 1.065 g/cm³. In another embodiment, the separation medium of the inventive method has a specific gravity between 1.065 to about 1.085 g/cm³, and preferably a specific gravity of about 1.070 to about 1.080 g/cm³, and an optimal specific gravity of 1.077 g/cm³. In a still further embodiment, the sodium citrate anticoagulant pH is from about 6.5 to about 7.5 and preferably 6.85 to about 7.15, and an optimal pH of 7.0;

In one embodiment, the concentration of sodium citrate anticoagulant is from about 0.05M to about 0.20M, with a preferred concentration of sodium citrate from about 0.08M to about 0.13M, with an optimal sodium citrate concentration range from about 0.09M to about 0.11M. In a further embodiment, the pH is 7.0 and the concentration of sodium citrate to 0.1M;

In a further embodiment, the anticoagulant comprises per liter 0.10 Molar 294 gm Sodium Citrate·2H2O, 0.27 gm Citric Acid·H₂O; and pH 7.0. In a certain embodiment, the coagulation-activator is a calcium chloride (CaCl₂·2H₂0) clot activation solution at a concentration of between 0.05M to 0.3M, preferably between 0.1M to approximately 0.25M, with an optimal concentration of 0.2M.

In another embodiment of the invention, provided is a purified non-naturally occurring wound healing composition, comprising platelets, monocytes and lymphocytes, said composition being substantially free of neutrophils, made by the method according to the above method. In a further embodiment, the purified non-naturally occurring wound healing composition is a mononuclear-platelet rich fibrin matrix.

In a still further method according to the invention, provided is a method of treating a wound, comprising the step of administering the purified non-naturally occurring wound healing composition of the invention which comprises platelets, monocytes and lymphocytes, said composition being substantially free of neutrophils, to a subject in need thereof. The M-PRFM is removed from the second vial and applied directly to the wound bed. The wound is then wrapped in appropriate dressings and bandages to support the M-PRFM in the wound bed for a period of several days.

In accordance with another embodiment of the present invention, a sodium citrate-based anticoagulant solution is prepared in the following manner. Trisodium citrate·2H₂O and citric acid·HO are dissolved in water in amounts sufficient to yield a sodium citrate solution having a desired concentration and pH which fall within the ranges set forth below. For example, a 0.1M sodium citrate solution having a pH of 7.0 (+0.15) can be prepared by dissolving 29.4 grams of Na citrate·2H₂O and 0.27 grams of citric acid·HO in a liter of H₂O. The concentration of the sodium citrate-based solution should be sufficient for preventing coagulation of a blood sample either added to a blood separation/collection device or involved in some other laboratory/clinical technique. In particular, the concentration of sodium citrate should range from about 0.05M to about 0.2M, and preferably from about 0.08M to about 0.13M. The most preferred range is from about 0.09M to about 0.11M. Additionally the pH of the final sodium citrate-based solution ranges from above pH 6.0 to about pH 8.5, and preferably from about pH 6.5 to about pH 7.5. In the most preferred embodiment, the pH ranges from about pH 6.85 to about pH 7.15, and ideally pH 7.0.

The anticoagulant solution may alternatively comprise, for example, ethylenelendiaminetetraacetic acid disodium salt, ethylenelendiaminetetraacetic acid dipotassium salt and tripotassium and combinations thereof. Other suitable citrate-based anticoagulant formulations include, for example, buffered sodium citrate comprising, for example, Per Liter: 0.109 Molar 0.129 Molar, 24.7 gm 32.0 gm Sodium Citrate·2H2O, and 4.42 gm 4.2 gm Citric Acid·HO, with a pH 6.1; Acid Citrate Dextrose (ACD-A and ACD-B) comprising, for example, Per Liter: 0.2 gm 0.2 gm Potassium Sorbate (Antimycotic), 220 gm 13.2 gm Sodium Citrate·2H2O, 24.5 gm. 14.7 gm Dextrose·HO, and 8.0 gm 4.8 gm Citric Acid·HO, with pH 5.05(ACD-A); pH 5.1 (ACD-B); Citrate Phosphate Dextrose (CPDA-1 Contains 0.275 gm Adenine) comprising, for example, Per Liter: 0.2 Potassium Sorbate, 26.3 gm Sodium Citrate·2H₂O, 3.27 gm Citric Acid·HO, 2.22 gm Monobasic Sodium Phosphate·H₂O 25.5 gm. Dextrose·HO, with a pH 5.8; Alsever's Solution comprising, for example, Per Liter: 8.0 gm Sodium Citrate·2H2O, 22.6 gm Dextrose·H₂O 4.2 gm Sodium Chloride, and Citric Acid to adjust pH to 6.1; and M-PRFM Citrate comprising, for example, Per Liter: 0.10 Molar, 294 gm Sodium Citrate·2H2O, and 0.27 gm Citric Acid·H₂O, with pH 7.0.

Once the solution of the present invention has been prepared according to the aforementioned steps, the solution may be either employed in some laboratory technique or added to any of several blood separation and/or collection tubes available in the art for separating lymphocytes, monocytes, and platelets from heavier phases of whole blood or a pre-treated cell fraction thereof. While the sodium citrate-based anticoagulant solution of the present invention can be used for any blood separation device, it affords the greatest advantages when used with those devices utilizing a thixotropic gel layer employed either as a cell density separation medium or as a barrier means for isolating various components of the device prior to centrifugation. In particular, the solution of the present invention can be employed in the construction of an improved blood separation assembly. The preferred embodiment of the assembly includes a container having a closed end and an open end. The container is preferably of the type known in the art capable of collecting a blood sample and undergoing subsequent centrifugation for separation of the sample.

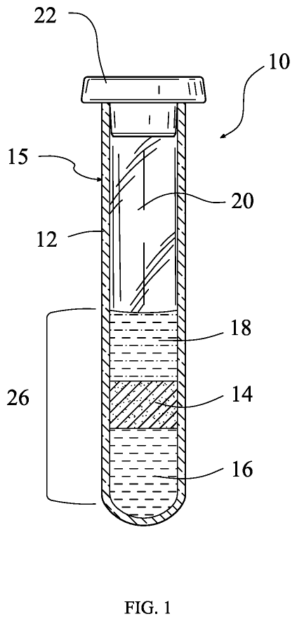

Referring to FIG. 1, a blood collection and separation system 10 is shown according to an exemplary embodiment of the invention. The system 10 includes a container or tube 12 and a layer of a high-viscosity, low-density immiscible gel 14, such as for example, a thixotropic gel, positioned within the tube at a first position, and a layer of a liquid density separation medium 16 positioned immediately below the thixotropic gel 14, with a layer of an anticoagulant solution 18 positioned above the gel 14. Reference number 26 denotes collectively the layered section comprising the anticoagulant solution 18, thixotropic gel 14, and liquid density separation medium layer 16. Typically, the liquid density separation medium 16 will be of a suitable type known in the art for separating mononuclear cells and platelets from whole blood, such as, for example, the commercially available liquid density-gradient separation medium Ficoll® Paque.

A variety of thixotropic gels known in the art may be used for the thixotropic gel 14 depending upon the desired operation to be performed. For example, if the thixotropic gel is employed as a separation medium as well as a barrier means, the gel should have a specific gravity between 1.055 g/cm3 to about 1.080 g/cm³, and preferably a specific gravity of about 1.060 g/cm3 to about 1.065 g/cm³. If the thixotropic gel is employed in conjunction with a liquid density gradient material, the gel primarily functions as a temporary barrier means prior to centrifugation. In such an assembly, the gel maintains isolation of a blood sample delivered to the tube from the liquid density gradient material residing in the tube until analysis can be performed at a later time. In such a situation, the specific gravity of the thixotropic gel should be within a sufficient range for allowing adequate separation of the mononuclear and platelet cell layers from the other components of the blood sample. Preferably, the thixotropic gel employed in such an assembly has a specific gravity ranging from about 1.055 g/cm3 to about 1.075 g/cm³. Thixotropic gels are well-known in the art and are typically water insoluble and chemically inert to blood. They are commonly formulated from a dimethyl polysiloxane or polyester and a precipitated methylated silica, wherein the methylation renders the material hydrophobic. The preferred embodiment of the improved blood separation assembly of the present invention also includes a suitable liquid density separation medium employed within the container at a second position which is further away from the open end of the container than is the thixotropic gel layer.

An exemplary method according to one embodiment of the invention includes the introduction of a sample of whole blood or a pretreated cell fraction of blood to the container 12 containing the layered section 26. Then, upon the subsequent centrifugation of assembly 10, the thixotropic gel layer 14 migrates from the first position toward the top end 15 of container 12. As centrifugation proceeds, the red blood cells and granulocytes separate from the mononuclear cell and platelet fraction and become concentrated in a layer immediately above the thixotropic gel. As the thixotropic gel moves toward a new position within the tube, the red blood cells and granulocytes migrate through the gel to displace the liquid density separation medium below. As the liquid density separation medium is displaced, it moves upward through the thixotropic gel to mix with the mononuclear cell and platelet fraction/anticoagulant solution. Red blood cells and granulocytes are pelleted toward the bottom of the tube while the lymphocytes, monocytes, and platelets form a highly purified mononuclear-platelet cell layer immediately above the thixotropic gel layer, thereby facilitating isolation and subsequent removal of the mononuclear and platelets cells.

Finally, the anticoagulant solution 18 is positioned above the thixotropic gel layer 14 so that it may adequately contact a whole blood sample introduced into the tube for centrifugation and subsequent isolation of the mononuclear and platelet cell layer. In FIG. 1, anticoagulant solution 18 is shown positioned above thixotropic gel layer 14 in closer proximity to the open end of the separation tube 10 than the gel. This anticoagulant solution 18 may have, for example, an effective concentration of sodium citrate sufficient for preventing coagulation of a sample of blood when such sample is later added to the tube for centrifugation and subsequent analysis. The anticoagulant solution may have a pH ranging from above pH 6.0 to about pH 8.5, and preferably from about pH 6.5 to about pH 7.5. Most preferably, the pH of the solution should range from about pH6.85 to about pH7.15. Optimally, the pH should be 7.0. In one embodiment of the improved blood separation 10, the concentration of sodium citrate should range from about 0.05M to about 0.2M, and preferably from about 0.08M to about 0.13M. Most preferably, the concentration of sodium citrate should be from about 0.09M to about 0.11M. Optimally, the concentration should be 0.1M.

While the anticoagulant solution suitable for use as the solution 18 is primarily composed of sodium citrate, additional reagents may be added, such as cell-sustaining solutions or other reagents, to provide additional properties to the solution. The preferred embodiment of the improved blood separation assembly of the present invention also includes a free space adjacent to the open end of the container or tube which is of a sufficient volume to receive a sample of whole blood or a fraction thereof, either alone or in conjunction with an added reagent. FIG. 1 shows free space 20 positioned above anticoagulant solution 16 to provide suitable space for accommodation of a blood sample to be separated.

Additionally, the assembly of the present invention may optionally include a closure element for sealing the open end of the container or tube. Typically, the closure element will be suitable for providing vacuum sealing of the open end of the container as well as being pierceable by a needle to adapt the container for drawing a sample of blood from a test subject. In FIG. 1, a closure element 22, such as for example, a seal is provided in the open end of the container or tube for creating a vacuum sealing of the container as mentioned above. Upon the addition of a blood sample to the assembly 10, mixing of the sample with the anticoagulant solution 18 occurs, typically by manual inversion of the container 12. The thixotropic gel layer 14 remains in a temporarily fixed, first position in the tube to serve as a barrier for isolating the blood sample/anticoagulant solution suspension from any contact with the other components of the assembly, such as the liquid density separation medium 16.

The present invention further includes a method for separating lymphocytes, monocytes, and platelets from heavier phases of a sample of whole blood or a pretreated cell fraction thereof. The method includes the steps of providing a container having an open and a closed end. Preferably, the container is a blood collection/separation tube of the type mentioned above. The method includes introducing a first layer of a thixotropic gel-like substance into the container or tube at a first position. The method further includes introducing an anticoagulant solution into the container at a second position in closer proximity to the open end of the container than the first thixotropic gel layer. The method also includes the steps of limiting the pH of the solution to any of the preferred ranges previously mentioned as well as the step of introducing the anticoagulant solution of the present invention into the container with an effective concentration of sodium citrate sufficient for preventing coagulation of a blood sample. Additionally, the method includes the step of limiting the concentration of sodium citrate in the solution to any of the ranges previously mentioned in the description of the anticoagulant solution.

The method of the present invention also includes the step of introducing a sample of whole blood or a pretreated cell fraction of blood into the container and the subsequent step of centrifuging the container to induce separation of lymphocytes, monocytes, and platelets from heavier phases of the sample.

Many methods and systems require the transfer of a fluid from one container to another. A common practice is to remove closures on two containers and to pipette liquid in one container to the other. This practice, however, exposes the sample to environmental contaminants. For example, this technique is used to transfer plasma that has been separated from red blood cells in a blood sample. A different technique is often required, however, to remove the plasma at the interface meniscus. Frequently the high-density, undesirable, lower-fraction red blood cells contaminate the aspirated sample. To avoid this problem, the pipette may be maintained a safe distance from the meniscus (i.e., the interface between the plasma and red blood cells), which could result in an incomplete transfer of the sample. The incomplete transfer of the desirable fraction results in lower than optimum volume yield and non-stoichiometric ratios of the sample reagents and those in the second container. This second condition can be a serious source of performance variation of the product. This is the case in many enzyme reactions in which reaction rates are a maximum at certain stoichiometric ratios and rapidly diminish at higher or lower ratios. Using a sterile needle and syringe does not improve the ability to recover various layers of plasma or cells, since the technique is still very sensitive to the operator's ability to accurately see the various layers and their experience.

Wound care is one of the most important issues in medicine, especially with respect to chronic ulcers. This issue is important not only because of the high cost of management, but also because of the variable success rate. Other problems associated with wound care include loss of liquids and the possibility of infections occurring. Synthetic or animal-origin membranes have been used in wound care as a dressing or to separate bone cavities from soft tissues in the process of re-ossification.

One treatment for wound care may include applying biological tissues or sponges (generally protein based) of animal origin, e.g., collagen, fibrin, albumin to a wound site. However, allergic and immunological responses are common with these applications. Most of these cases are not resolved with a single application and may require multiple applications.

Another treatment includes skin transplantation, which is performed for the most difficult cases. Skin transplantation is expensive, however, and significantly increases overall treatment costs. A mesh of modified animal collagen is used to support the new autologous tissue. The application is a difficult process that may take up to 20 days for cultivation of dermal tissue, with the possibility of contamination the device.

Overall, methods and systems for preparing autologous PRP, or M-PRP, or a solid fibrin matrix which is capable of regenerating tissue in a living organism are desired.

The present invention also provides systems and methods for forming a solid-fibrin matrix or autologous fibrin membrane capable of regenerating tissue in a living organism. In these methods and systems, anticoagulated plasma containing mononuclear cells and platelets is obtained by centrifugation of a blood sample. The transfer device described herein enables the cell-plasma suspension to be transferred to a second container containing calcium-clotting agents and then immediately centrifuged to obtain a stable, dense, autologous fibrin mononuclear-platelet network. The transfer devices described herein may also be used to transfer other liquids in other applications. In other words, the methods, transfer devices and systems described herein enable concurrent centrifugation and coagulation.

By using these systems and methods, at least one of the following may be achieved: 1) the sample is manipulated in a manner by which sterility is maintained; 2) the total volume of plasma is transferred to maximize a full yield of a clot; 3) the stoichiometric ratio of anticoagulant and calcium clotting agent is maintained in a narrow range to minimize clotting time; 4) the pH of the applied matrix is close to the normal pH of human tissue, thus avoiding a stinging sensation, 5) the transfer is completed quickly and can be performed inter-operatively within the half-life of the platelet-derived growth factors; 6) health care providers not normally performing these operations (e.g., Nurse Practitioner) can easily perform these methods and operate the systems; and 7) the devices are single use to prevent re-use and possible contamination by blood-borne pathogens.

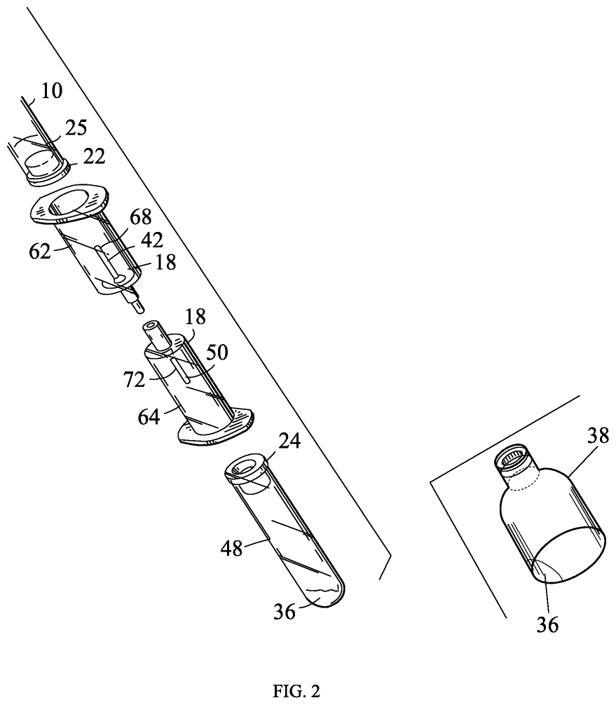

Generally speaking, the present invention provides integrated systems and methods for preparing a solid-fibrin matrix or autologous fibrin membrane which can be used to regenerate tissue in a living organism. In one embodiment depicted in FIG. 2, the system comprises a primary container 10, such as the container 10 of FIG. 1, a secondary container 48 (or an alternative container 38) and a transfer device 18. Preferably, the primary and secondary containers 10, 48 are tubes, or a flat bottom vial 38, and more particularly, test tubes or vials, although any container that can hold a fluid or liquid and can be centrifuged is suitable for use with the invention. Preferably, the containers 10, 48, and 38 are made from glass or plastics.

The primary container 10 should be capable of drawing blood therein using standard venipuncture techniques. Preferably the primary container 10 is sealed with a seal 22 while the blood is being drawn to prevent contamination, although the container 10 may be sealed shortly thereafter. A variety of seals 22 can be used to seal the primary container 10, e.g., a rubber stopper, cap, foam, elastomer or other composite. The seal 22 should be capable of being pierced or punctured, and therefore rubber and silicone are preferred materials from which the seal is fabricated, although any material that provides a seal and is capable of being pierced can be used. The primary container 10 may contain the layered section 26 of FIG. 1.

In operation, the anticoagulant 18 tends to slightly dilute the blood collected in the primary container 10 to place it in condition for centrifugation. In addition, the primary container includes a density-gradient separation medium 26, air 27 as well as a high-viscosity, low-density gel 28.

The transfer device 18 may comprise two pieces as depicted in FIG. 2. The transfer device 18 comprises a cannula having a first end 42 having a first opening and a second end 50 having a second opening. The ends 42, 50 of the cannula are sharp or pointed (or even have a bevel ground on them) to be able to puncture or penetrate the seals 22, 24 of the primary and secondary containers 10, 48 or 38. The cannula is recessed and coaxially mounted within the housing to prevent accidental finger stick during manipulation of the containers. The housing 58 has two cylindrical, opposed guides 62, 64 which are centrally and axially oriented with the cannula. The guides 62, 64 serve to guide the primary 10 and secondary containers 48 or 38 onto the first and second ends 42, 50 of the transfer device 18.

The ends 42, 50 of the cannula may be encompassed or covered by safety valves, sheaths or elastomeric sleeves 68, 72, which form a hermetic seal. The safety sheaths 68, 72 also cover the first and second openings 46, 54. When the first and second ends 42, 50 puncture the elastomeric sleeves 68, 72, the sleeves 68, 72 retract accordingly. The ends 42, 50 extend far enough to fully puncture the seals 22, 24, but not extend much further into the containers 10, 48, or 38. This allows maximum volume transfer of the inverted primary container's 10 liquid volume to the secondary container 48 or 38. The elastomeric sleeves 68, 72 prevent the flow of gas or liquid when not punctured. Suitable materials for the sleeves 68, 72 include, but are not limited to, rubber varieties and thermoplastic elastomers.

Subsequent to centrifugation of the blood collection tube 10, the sealed primary holder 10 is inverted before the transfer device 18 is used to puncture the seal 22. In other words, the primary container 10 is inverted such that the sealed opening is in the lowest vertical position. Inverting the primary container changes the order in which the layers are arranged. Above the seal 22 are the following layers in sequence from bottom to top: the mononuclear-platelet-rich plasma, the gel 14 (shown in FIG. 1), the residual gas, the separation medium 16 and the red blood cells.

Next, the secondary container 48 or 38 is placed in a vertical position with its sealed opening 24 in the topmost position as shown in FIG. 2. This positions the secondary container 48 or 38 for the transfer of the primary holder's contents therein. The transfer device's guide 64 is then placed over and guides the secondary container 48 or 38 therein, while the inverted primary container 10 is then placed into the other guide 62 (or vice versa). In other words, either end 42, 50 of the cannula can be used to puncture either seal 22, 24. Because the transfer device 18 is symmetrical on either end, the user is provided a degree of foolproof operation. The user then forces the containers together to puncture both seals 22, 24 with each respective cannula end 42, 50. The two valve sleeves 68, 72 covering the ends 42, 50 further enhance the foolproof operation. First, if the first end 42 punctures the primary seal 22 (again, either end can be used to puncture either seal), the unpunctured sleeve 72 covering the other end 50 will contain the fluid, thereby preventing the fluid from spilling. On the other hand, if the other end 50 punctures the other seal 24 (and the sleeve 72 accordingly) first, the vacuum is maintained by the sleeve 68 covering the first end 42.

Once the ends 42, 50 puncture both sleeves 68, 72 and seals 22, 24 as shown the desired fluid is transferred from the primary container 10 to the secondary container 48, or 38 if it is used, by pressure differential. In other words, because the pressure in the secondary container 48 or 38 has been evacuated, the contents (more particularly, the mononuclear-platelet rich plasma) of the primary container 10 flow into the secondary container 48 or into 38 if it is used. The pressure in the primary container 10, originally at atmospheric, decreases as the liquid level diminishes and the gas volume expands. At no point, however, is the pressure equal to zero. Because the secondary container 48 or 38 is fully evacuated to a pressure equal to or slightly greater than zero, the pressure therein does not increase as the tube is filled since there is little or no gas to compress.

Because of the sequential arrangement of the layers in the primary container 10, the mononuclear-platelet-rich plasma is easily transferred to the secondary container 48 or 38 via the transfer device 18. In addition, because the primary container 10 is also preset to an evacuation level, the container only partially fills after blood collection. This allows the gas in the “head space” to remain significantly above zero during transfer when its volume is expanded, thereby allowing fast and complete transfer to the secondary container 48 or 38, if used. This is dictated by the ideal gas law and the Poiseuille-Hagen equation.

The transfer of the mononuclear-platelet-plasma fraction to the secondary container 48 or 38 is complete, thereby allowing maximum yield and maintenance of the appropriate stoichiometric ratio of reagents. The mononuclear-platelet-plasma then contacts the coagulation activator 36 in the second container 48 or in 38, thereby creating a mixture which can be immediately centrifuged to form a solid-fibrin web. The pressure differential between primary and secondary containers 10 and 48 or 38 is substantially maintained throughout transfer, allowing rapid transfer. The transfer device 18 is unaffected by order of tube engagement, rendering the system virtually foolproof. Finally, the transfer may occur without venting, maintaining sterility and non-contamination of the sample.

In the secondary container 48 or 38, the mononuclear-platelet-plasma suspension contacts the calcium-coagulation activator 36, immediately after which concurrent coagulation and centrifugation of the plasma can take place to form the solid-fibrin web. The solid-fibrin web is suitable for regenerating body tissue in a living organism. Such a method alleviates the need to first pre-concentrate the plasma by removing water therefrom before the plasma is contacted with the calcium-coagulation activator 36. In addition, the transfer device 18 can be used to transfer blood or other fluids in a wide variety of application.

The use of the secondary container 48 allows the resulting mononuclear-platelet fibrin matrix formed following centrifugation to form in the shape of the secondary tube 14 bottom, thus forming a mononuclear-platelet rich fibrin matrix (M-PRFM). Alternatively, using the secondary vial 38, following centrifugation, the resulting mononuclear-platelet rich fibrin matrix forms a M-PRFM membrane with the same diameter as the secondary vial 38.

In a further embodiment, the invention also provides a ready-to-use kit which comprises the primary container(s) 10, the secondary container 48 or 38, the transfer device 18, an alcohol swab to cleanse the venipuncture site, a multiple sample blood collection needle (21 gauge×1″), a safety holder, an elastic bandage, and a sterile culture dish to receive the M-PRFM matrix or membrane. The components can be arranged in a wide variety of manners within the kit.

The invention will now be further described in the Examples below, which are intended as an illustration only and do not limit the scope of the invention.

### EXAMPLES

### Example 1

The separation of mononuclear cells and platelets from whole blood achieves a relatively enriched cell-plasma suspension. A reproducibility study of percent recovery, percent purity, percent viability, red blood cell contamination, and granulocyte contamination was conducted using evacuated blood collection tubes containing a density gradient fluid, a thixotropic gel, and a citrate anticoagulant. Ten blood samples from one donor were collected, approximately 8.0 mL per tube, which contained 1.0 mL of a citrate anticoagulant with pH 7.0. The tubes were centrifuged for 20 minutes at 1500×g. The resulting mononuclear-platelet fraction was inverted 7 times to resuspend the cells into the plasma to create a Mononuclear-Platelet Rich Plasma (M-PRP). The M-PRP was assayed in duplicate using an automated hematology analyzer. No final washing steps were performed. The samples were resuspended to approximately equal final volumes. Between tube variation was calculated by differencing the mean of the duplicate readings for each tube.

TABLE 1

| Parameter | Mean % | SD | CV % |
|---|---|---|---|
| Recovery | | | |
| Mononuclear Cells | 70.3 | 9.7 | 13.8 |
| Platelets | 75 | 5.5 | 7.5 |
| Purity of Recovered Cells | | | |
| Lymphocytes | 84.4 | 1.3 | 1.5 |
| Monocytes | 13.9 | 1.4 | 10.4 |
| Viability | 100 | 0 | 0 |
| RBC Contamination | 7.4 | 1.6 | 21.7 |
| PMN Contamination | 1.7 | 0.6 | 34.5 |

Table 1. Reproducibility study of percent Recovery, percent Viability, percent RBC and percent PMN (polymorphonuclear cells or granulocytes) contamination from whole blood using an evacuated cell separation device containing a density gradient liquid, a thixotropic gel, and a citrate anticoagulant (pH 7.0).

### Example 2

The whole blood specimen is collected using the blood collection tube containing the sodium citrate anticoagulant, the thixotropic gel, and the density gradient liquid. The tube is gently inverted seven times to mix the anticoagulant with the whole blood. The tube is placed in a swing-out bucket rotor and centrifuged at 1500×g for 20 minutes. Once the M-PRP is separated from the whole blood specimen in the gel separation tube, the M-PRP is aseptically transferred to the second vial containing the sodium citrate. The solutions are gently mixed and the second vial is centrifuged for 25 minutes at 3500×g. Upon completion of the second centrifugation step, the vial is uncapped and the M-PRFM is recovered by inverting the vial to empty the contents onto a sterile receiving device such as a tissue culture dish. The M-PRFM is retrieved by sterile forceps, for example, and placed directly onto the wound bed. The wound is then covered with the appropriate coverings and bandages to support the M-PRFM in the wound for several days. The procedure can be repeated weekly as needed.

### REFERENCES

Demidova-Rice, T N, et al. (2012) Adv Skin Wound Care. 25(7): 304-14
Sibbald, R G, et al. (2011) Adv Skin Wound Care. 24: 415-37
Cañedo-Dorantes, L, et. al. (2019) Int J Inflam. 2019: 3706315
Gould, L, et al. (2015) Wound Repair Regen. 23(1): 1-13
Eming, S A, et al. (2014) Sci Transl Med. 6(265): 265sr6
Moulik, P K, et al. (2003) Diabetes Care. 26(2): 491-494
Armstrong, D G, et al. (2007) Int. Wound J. 4(4): 286-287
Nussbaum, S R, et al. (2018) Value Health. 21(1): 27-32
Barakat, M, et al. (2020) Adv Wound Care. https://doi.org/10.1089/wound2020.1254
Lefrançais, E, et al. (2017) Nature. 544(7648): 105-109
Etulain J. (2018) Platelets. 29(6): 556-568
Eisinger, F, et al. (2018) Front Med. 5: 317
Blair, P, et al. Blood Rev. (2009) 23(4): 177-189
Cloutier, N, et al. (2018) PNAS 115(7): E1550-E1559
Neumüller, J, et al. In: Khan M, ed. Intechopen. London: Intechopen; (2015). pp. 255-284
Hu H, et al. Thromb Haemost (2010) 104: 1184-1192
Miyamoto, H, et al. J Oral Max Surgery, Med, and Path. (2020) 32(4): 237-240
Langer H F, et al. Arterioscler Thromb Vasc Biol. (2007) 27: 1463-1470
Massberg S, et al. J Exp Med. (2006) 203: 1221-1233
Langer H F, et al. J Mol Cell Cardiol. (2009) 47: 315-325
Boulaftali Y, et al. J Clin Invest. (2013) 123: 908-916
Xu X, et al. Cells Tissues Organs. (2013) 197: 103-113
Pietrzak, W S, et al. (2005) J Craniofac Surg. 16: 1043-1054
Frykberg, R G, et al. (2010) Ostomy Wound Manage. 56(6): 36-44
Zhou, Y, et al. (2016) BioMed Res. Int. 2016: 1-8
Fedorova, N V, et al. (2018) Mediat Inflamm. 2018: ID 1574928
Wong, S L, et al. (2015) Nat Med. 21(7):815-819
Wier, E, et al. bioRxiv (2020.07.06). 189910
Mosser D M, et al. (2008) Nature Rev Immunol. 8(12): 958-969
Porta, C, et al. (2015) Semin Immunol. 27(4): 237-248
Abramson, S L, et al. (1990) J Immunol. 144(2): 625-630
Anders, C B, et al. (2019). Curr Opinion Infect Dis. 32(3): 204-209
Jin, E, et al. (2013) J Cell. Mol. Med. 17(12): 1644-1651
Quiros, M, et al. (2017) J Clin Invest. 127(9): 3510-3520
Wang, X, et al. (2019) Adv in Wound Care 8(11): 527-537
Park, J E, et al. Am J Surg. (2004) 187(5): S11-S16
Fitzpatrick, J, et al. (2017) Orthop J Sports Med. 5(1):2325967116675272
Margolis, J. (1956) Nature. 178: 805-806

It is to be understood that the invention is not limited to the particular embodiments of the invention described above, as variations of the particular embodiments may be made and still fall within the scope of the appended claims.

1. A system for preparing a purified non-naturally occurring wound-healing composition comprising platelets, monocytes, and lymphocytes, and substantially free of neutrophils the system comprising:
a first container having a sealable open end for receipt of a blood sample, and a closed end opposite the open end;
the first container containing a density separation medium disposed at a first position proximate the closed end, and a thixotropic gel having a density of approximately 1.055 to 1.080 g/cm3 disposed at a second position between the open end and the first position; and an anticoagulant solution having a pH in the range of 6.0 and 8.5 at a third position between the open end and the second position,
wherein the first container is useable to produce an extractable suspension of separated platelets, monocytes, lymphocytes and plasma from the blood sample when centrifuged; and
a second container having a sealable open end for receipt of an extracted suspension of separated platelets, monocytes, lymphocytes and plasma produced in the first container, the second containing a coagulation-activator,
wherein the second container is useable to produce the wound-healing composition from the suspension of separated platelets, monocytes, and lymphocytes when centrifuged.

2. The system of claim 1, further comprising a transfer device adapted for coupling with the first container open end, and the second container open end, wherein the transfer device when coupled to the first container open end creates a sterile seal for receipt of the suspension of separated platelets, monocytes, lymphocytes, and plasma from the first container, and when coupled to the second container open end creates a sterile environment for transfer of the suspension of separated platelets, monocytes, lymphocytes, and plasma to such second container.

3. The system of claim 1, wherein said density separation medium comprises at least one of a non-Newtonian gel and a Newtonian liquid.

4. The system of claim 1, wherein said density separation medium comprises an ionic substance having a molecular weight of less than about 1500.

5. The system of claim 4, wherein said density separation medium is selected from the group consisting of sodium diatrizoate, derivatives thereof and combinations thereof.

6. The system of claim 1, wherein said density separation medium is selected from the group consisting of a polymer of sucrose or epichlorohydrin having a molecular weight of at least 400,000 and derivatives and combinations thereof.

7. The system of claim 1, further comprises a first closure device for sealing the open end of the first container.

8. The system of claim 7, wherein the first closure device is adapted for vacuum sealing said open end of the first container.

9. The system of claim 8, wherein the first closure device is pierceable by a cannula for supplying the blood sample to the first container by pressure differential.

10. The system of claim 1, further comprises a second closure device for sealing the open end of the second container.

11. The system of claim 10, wherein the second closure device is adapted for vacuum sealing said open end of the second container.

12. The system of claim 11, wherein the second closure device is pierceable by a cannula for supplying the suspension of separated platelets, monocytes, lymphocytes, and plasma to the second container by pressure differential.

13. The system of claim 1, wherein the purified wound-healing composition comprising platelets, monocytes, and lymphocytes is a mononuclear-platelet rich fibrin matrix.

14. The system of claim 1, further comprising a centrifuge adapted to receive at least one of the first and second containers.

15. The system of claim 1, wherein the thixotropic gel has a specific gravity of between 1.060 to about 1.065 g/cm³.

16. The system of claim 1, wherein the separation medium has a specific gravity between 1.065 to about 1.085 g/cm³, and preferably a specific gravity of about 1.070 to about 1.080 g/cm³, and an optimal specific gravity of 1.077 g/cm³.

17. The system of claim 1, wherein said pH is from about 6.5 to about 7.5 and preferably 6.85 to about 7.15, and an optimal pH of 7.0.

18. The system of claim 1, wherein the concentration of sodium citrate anticoagulant is from about 0.05M to about 0.20M, with a preferred concentration of sodium citrate from about 0.08M to about 0.13M, with an optimal sodium citrate concentration range from about 0.09M to about 0.11M.

19. The system of claim 1, wherein pH is 7.0 and the concentration of sodium citrate to 0.1M.

20. The system of claim 1, wherein the anticoagulant comprises per liter 0.10 Molar 294 gm Sodium Citrate·2H₂O, 0.27 gm Citric Acid·H₂O; and pH 7.0.

21. The system of claim 1, wherein the anticoagulant comprises at least one of ethylenelendiaminetetraacetic acid disodium salt, ethylenelendiaminetetraacetic acid dipotassium salt or tripotassium salt.

22. The system of claim 1, wherein the coagulation-activator is a calcium chloride (CaCl₂·2H₂0) clot activation solution at a concentration of between 0.05M to 0.3M, preferably between 0.1M to approximately 0.25M, with an optimal concentration of 0.2M.

23. A system for preparing a purified non-naturally occurring wound-healing composition comprising platelets, monocytes, and lymphocytes, and substantially free of neutrophils the system comprising:
a first container having a sealable open end for receipt of a blood sample, and a closed end opposite the open end;
the first container containing a first density separation medium disposed at a first position proximate the closed end, and a second density separation medium disposed at a second position between the open end and the first position; and an anticoagulant solution having a pH in the range of 6.0 and 8.5 at a third position between the open end and the second position,
wherein the first container is useable to produce an extractable suspension of separated platelets, monocytes, lymphocytes, and plasma from the blood sample when centrifuged; and
a second container having a sealable open end for receipt of an extracted suspension of separated platelets, monocytes, lymphocytes, and plasma produced in the first container, the second containing a coagulation-activator,
wherein the second container is useable to produce the wound-healing composition from the suspension of separated platelets, monocytes, lymphocytes, and plasma when centrifuged.

24. The system of claim 23, wherein the second density separation medium is a thixotropic gel having a density of approximately 1.055 to 1.080 g/cm³.

25. The system of claim 23, wherein said first density separation medium comprises at least one of a Newtonian liquid.

26. The system of claim 23, wherein said first density separation medium comprises an ionic substance having a molecular weight of less than about 1500.

27. The system of claim 26, wherein said first density separation medium is selected from the group consisting of sodium diatrizoate, derivatives thereof and combinations thereof.

28. The system of claim 23, wherein said first density separation medium is selected from the group consisting of a polymer of sucrose or epichlorohydrin having a molecular weight of at least 400,000 and derivatives and combinations thereof.

29. The system of claim 23, wherein the purified wound-healing composition comprising platelets, monocytes, and lymphocytes is a mononuclear-platelet rich fibrin matrix.

30. A purified non-naturally occurring wound healing composition, comprising platelets, monocytes, and lymphocytes, wherein said composition is substantially free of neutrophils.

31. The composition according to claim 30, wherein the concentration of neutrophils is less than 5% of the separated white blood cells.

32. The purified non-naturally occurring wound healing composition according to claim 30, wherein the composition is a mononuclear-platelet rich fibrin matrix.

33. A method of treating a wound, comprising the step of administering the purified non-naturally occurring wound healing composition according to claim 30 to a subject in need thereof.

34. A method of producing a purified non-naturally occurring wound healing composition comprising platelets, monocytes, lymphocytes, and plasma from a sample of blood, comprising the steps of:
introducing said sample of blood into a first container having a sealable open end for receipt of said sample, and a closed end opposite the open end, said first container containing a density separation medium disposed at a first position proximate the closed end, and a thixotropic gel having a density of approximately 1.055 to 1.080 g/cm3 disposed at a second position between the open end and the first position; and an anticoagulant solution having a pH in the range of 6.0 and 8.5 at a third position between the open end and the second position;
centrifuging said first container to produce an extractable suspension of separated platelets, monocytes, lymphocytes, and plasma from the blood sample;
extracting said extractable suspension of separated platelets, monocytes, lymphocytes, and platelets into a second container containing a coagulation-activator and having a sealable open end for receipt of said extracted suspension of separated platelets, monocytes, lymphocytes, and plasma produced in the first container; and
centrifuging said second container to produce the purified non-naturally occurring wound healing composition comprising platelets, monocytes, and lymphocytes.

35. The method according to claim 34, wherein the wound healing composition is substantially free of neutrophils.

36. The method according to claim 35, wherein the concentration of neutrophils is less than 5% of the separated white blood cells.

37. The method according to claim 34, wherein said density separation medium comprises at least one of a non-Newtonian gel and a Newtonian liquid.

38. The method according to claim 34, wherein said density separation medium comprises an ionic substance having a molecular weight of less than about 1500.

39. The method according to claim 38, wherein said density separation medium is selected from the group consisting of sodium diatrizoate, derivatives thereof and combinations thereof.

40. The method according to claim 34, wherein said density separation medium is selected from the group consisting of a polymer of sucrose or epichlorohydrin having a molecular weight of at least 400,000 and derivatives and combinations thereof.

41. The method according to claim 34, wherein the purified wound-healing composition comprising platelets, monocytes, and lymphocytes is a mononuclear-platelet rich fibrin matrix.

42. The method according to claim 34, wherein the thixotropic gel has a specific gravity of between 1.060 to about 1.065 g/cm³.

43. The method according to claim 34, wherein the separation medium has a specific gravity between 1.065 to about 1.085 g/cm³, and preferably a specific gravity of about 1.070 to about 1.080 g/cm³, and an optimal specific gravity of 1.077 g/cm³.

44. The method according to claim 34, wherein said pH is from about 6.5 to about 7.5 and preferably 6.85 to about 7.15, and an optimal pH of 7.0.

45. The method according to claim 34, wherein the concentration of sodium citrate anticoagulant is from about 0.05M to about 0.20M, with a preferred concentration of sodium citrate from about 0.08M to about 0.13M, with an optimal sodium citrate concentration range from about 0.09M to about 0.11M.

46. The method according to claim 41, wherein pH is 7.0 and the concentration of sodium citrate to 0.1M.

47. The method according to claim 34, wherein the anticoagulant comprises per liter 0.10 Molar 294 gm Sodium Citrate-2H₂O, 0.27 gm Citric Acid·H₂O; and pH 7.0.

48. The method according to claim 34, wherein the coagulation-activator is a calcium chloride (CaCl₂·2H₂O) clot activation solution at a concentration of between 0.05M to 0.3M, preferably between 0.1M to approximately 0.25M, with an optimal concentration of 0.2M.

49. A purified non-naturally occurring wound healing composition, comprising platelets, monocytes, and lymphocytes, said composition being substantially free of neutrophils, made by the method according to claim 34.
